(19) United States
(12) Patent Application Publication
(10) Pub. No.: US 2010/0184819 A1
(43) Pub. Date: Jul. 22, 2010

(54) METHOD FOR REDUCING INTRACRANIAL PRESSURE

(76) Inventor: Robert Vink, Glenelg (AU)

Correspondence Address: KLARQUIST SPARKMAN, LLP, 121 SW SALMON STREET, SUITE 1600, PORTLAND, OR, 97204, US

(21) Appl. No.: 12/669,462

(22) PCT Filed: Jul. 18, 2008
(86) PCT No.: PCT/AU2008/001033

§ 371 Date: Mar. 26, 2010

(30) Foreign Application Priority Data
Jul. 19, 2007 (AU) 2007903902

(51) Int. Cl. A61K 31/405; A61P 25/00

(52) U.S. Cl. 514/419

(57) ABSTRACT

The present invention relates generally to methods for reducing intracranial pressure in a subject. More particularly, the methods of the present invention include administering to the subject an effective amount of a substance P receptor antagonist.

### PRIORITY CLAIM

This international patent application claims priority to Australian provisional patent application 2007903902 filed 19 Jul. 2007, the contents of which are herein incorporated by reference.

### FIELD OF THE INVENTION

The present invention relates to a method for reducing intracranial pressure.

### BACKGROUND OF THE INVENTION

A rise in intracranial pressure (ICP) is a common, life-threatening complication associated with a large number of clinical conditions. It is the primary cause of death in acute neurological conditions such as stroke, traumatic brain injury, CNS infections and neoplasia. Raised ICP is also a major complication following neurosurgical intervention, and represents a major complication and leading cause of mortality with systemic conditions such as liver failure, chronic obstructive lung disease, malaria and altitude sickness.

Intracranial pressure is the pressure exerted by the contents of the cranium. The brain is encased within the skull, or cranium, which normally serves to protect the brain. The brain tissue itself, together with the other cranial contents of blood and cerebral spinal fluid normally exert a small positive pressure within the cranium, giving rise to the normal intracranial pressure observed. Normal intracranial pressure generally ranges from 0-15 mm Hg.

However, if there is an increase in the volume of any one of the components within the cranium, this will quickly lead to complications. The volume of the brain tissue may increase, for example, as a result of tumours, haemorrhaging, infections, cytotoxic or vasogenic oedema, and ischaemia. There can also be increases in the volume of cerebrospinal fluid (e.g. due to obstructive hydrocephalus) or blood within the cranium (e.g. due to for example high blood CO₂ levels, acidosis). Now the skull, which served to protect the brain, contributes to its injury. Because the cranium is a rigid, closed system, it has a very limited ability to accommodate changes in volume before a rise in ICP occurs. This now leads to a chain of events that are common, irrespective of the initial factor triggering the rise in ICP.

As ICP rises, blood perfusion and tissue oxygenation becomes difficult. In an attempt to compensate for this, arterial vasodilation occurs in order to increase perfusion, but this leads to intracranial hypertension, which further increases the ICP. As ICP continues to increase, it begins to approach arterial blood pressure. At this stage the brain tissue begins to experience hypercapnia, and the patient rapidly deteriorates with decreasing levels of consciousness, bradycardia, dilated and sluggish pupils. As the volume increases further, the brain begins to experience hypoxia and lactic acidosis, leading to further vasodilation and volume increases. As the pressure keeps rising one gets herniation (shifts) of the brain tissue, causing ischaemia in the herniated region. Finally, once ICP equals systolic arterial pressure, blood flow to the brain will cease and death will rapidly ensue.

Hence, an initial small rise in ICP can trigger a vicious cycle of events that may lead to death or permanent brain damage.

A critical aspect of the process leading to raised ICP are the vascular events that occur in response to either the initial trigger, or the initial rise in ICP. In terms of therapeutic intervention, osmotic diuretics, primarily mannitol, are used to try and lower ICP, but their effectiveness is questionable. Whilst, in theory, one would expect these agents to be able to extract fluid from brain tissue, their actual ability to do so effectively is limited. Indeed, controlled clinical trials have shown that there are no significant clinical benefits gained by the use of osmotic diuretics.

Other therapeutic interventions are primarily aimed at altering the balance between oxygen supply and demand in the brain. Basically, if it is not possible to increase blood and oxygen supply to the brain, the alternative strategy is to try and reduce the brain's need for oxygen by lowering its activity. A number of approaches may be used, including inducing a coma with barbiturates, or causing hypothermia.

The final alternative is surgical intervention, in the form of a decompressive craniectomy. In this procedure, part of the skull is surgically removed in order to relieve the pressure, and then is replaced a number of weeks-to-months later. This represents a very expensive, involved and protracted procedure, and is only occasionally done as a last-resort intervention.

Hence there is a very real clinical need for an effective therapeutic intervention that can help reduce both intracranial volume and pressure, preferably early in the cycle of events, and before other injury mechanisms occur. The present invention relates to a method for reducing intracranial pressure by administering to a subject a substance P receptor antagonist.

A reference herein to a patent document or other matter which is given as prior art is not to be taken as an admission that that document or matter was known or that the information it contains was part of the common general knowledge as at the priority date of any of the claims.

### SUMMARY OF THE INVENTION

The present invention arises from studies into the role of water channels associated with the cerebral vasculature and the role of substance P in modulating vascular function.

In the present studies it has been demonstrated that administration of antagonists of the substance P receptor are able to facilitate the movement of water from the brain, and thereby can reduce intracranial pressure in two animal models of brain trauma. Therefore, it is possible to reduce raised ICP, and thereby prevent or reduce the mortality and morbidity associated with this severe complication.

Accordingly, the present invention provides a method of reducing intracranial pressure in a subject, the method including administering to the subject an effective amount of a substance P receptor antagonist.

The present invention also provides use of a substance P receptor antagonist in the preparation of a medicament for reducing intracranial pressure in a subject.

The present invention also provides a method of preventing and/or treating raised intracranial pressure in a subject, the method including administering to the subject an effective amount of a substance P receptor antagonist.

The present invention also provides use of a substance P receptor antagonist in the preparation of a medicament for preventing and/or treating raised intracranial pressure in a subject.

The present invention also provides a method of improving blood flow to the brain in a subject suffering from raised intracranial pressure, the method including administering to the subject an effective amount of a substance P receptor antagonist.

The present invention also provides use of a substance P receptor antagonist in the preparation of a medicament for improving blood flow to the brain in a subject suffering from raised intracranial pressure.

The present invention also provides a method of improving oxygen delivery to the brain and/or improving oxygenation of the brain in a subject suffering from raised intracranial pressure, the method including administering to the subject an effective amount of a substance P receptor antagonist.

The present invention also provides use of a substance P receptor antagonist in the preparation of a medicament for improving oxygen delivery to the brain and/or improving oxygenation of the brain in a subject suffering from raised intracranial pressure.

The present invention also provides a method of reducing the risk of mortality or morbidity in a subject susceptible to, or suffering from, raised intracranial pressure, the method including administering to the subject an effective amount of a substance P receptor antagonist.

The present invention also provides use of a substance P receptor antagonist in the preparation of a medicament for reducing the risk of mortality or morbidity in a subject susceptible to, or suffering from, raised intracranial pressure.

The present invention also provides a method of reducing the risk of a neurological complication due to raised intracranial pressure in a subject, the method including administering to the subject an effective amount of a substance P receptor antagonist.

The present invention also provides use of a substance P receptor antagonist in the preparation of a medicament for reducing the risk of a neurological complication due to raised intracranial pressure in a subject.

The present invention also provides a method of improving movement of water from the brain of a subject into the circulation, the method including administering to the subject an effective amount of a substance P receptor antagonist.

The present invention also provides use of a substance P receptor antagonist in the preparation of a medicament for improving movement of water from the brain of a subject into the circulation.

The present invention also provides a method of improving the prognosis or outcome of a subject suffering from raised intracranial pressure, the method including administering to the subject an effective amount of a substance P receptor antagonist.

The present invention also provides use of a substance P receptor antagonist in the preparation of a medicament for improving the prognosis or outcome of a subject suffering from raised intracranial pressure.

Various terms that will be used throughout the specification have meanings that will be well understood by a skilled addressee. However, for ease of reference, some of these terms will now be defined.

The term “substance P receptor antagonist” as used throughout the specification is to be understood to mean an agent that directly or indirectly inhibits the binding of substance P to one of its receptors. Substance P is an excitatory neurotransmitter and is a peptide having the structure RPKPEEFFGLM-NH₂. Methods for determining the ability of an agent to act as a substance P receptor antagonist are known in the art.

While it will be appreciated that the substance P receptor antagonists of the present invention are agents that directly or indirectly inhibit the binding of substance P to one of its receptors, direct or indirect antagonists of substance P itself are also included within the scope of the invention.

The term “subject” as used throughout the specification is to be understood to mean a human or animal subject.

In this regard, it will be understood that the present invention also includes within its scope veterinary applications. For example, the animal subject may be a mammal, a primate, a livestock animal (eg. a horse, a cow, a sheep, a pig, or a goat), a companion animal (eg. a dog, a cat), a laboratory test animal (eg. a mouse, a rat, a guinea pig, a bird, a rabbit), an animal of veterinary significance, or an animal of economic significance.

The term “variant” as used throughout the specification is to be understood to mean an amino acid sequence of a polypeptide or protein that is altered by one or more amino acids. The variant may have “conservative” changes, wherein a substituted amino acid has similar structural or chemical properties to the replaced amino acid (e.g., replacement of leucine with isoleucine). A variant may also have “non-conservative” changes (e.g., replacement of a glycine with a tryptophan) or a deletion and/or insertion of one or more amino acids. The term also includes within its scope any insertions/deletions of amino acids for a particular polypeptide or protein. In some case the variant will be a “functional variant”. A “functional variant” will be understood to mean a variant that retains the functional capacity of a reference protein or polypeptide.

The term “prevent” as used throughout the specification is to be understood to mean an intervention that prevents the onset of a disease, condition or state in a subject. The term “treat” as used throughout the specification is to be understood to mean an intervention that improves the prognosis and/or state of a subject with respect to a disease, condition or state in a subject

### BRIEF DESCRIPTION OF THE FIGURES

FIG. 1 shows electron micrographs of perivascular AQP-4 immunostaining at 5 hr following diffuse TBI. AQP-4 channels can be clearly identified in sham (uninjured) control animals as an electron dense layer (arrowed) at the astrocyte end-foot processes surrounding blood vessels (A). At 5 h after injury (B), there is a profound reduction in AQP-4 electron density in vehicle-treated (placebo) animals. Isolated AQP-4 electron density is still visible (arrowed), however astrocyte swelling has become apparent (asterisks). Administration of an NK1 antagonist (NAT) at 30 min after injury resulted in a rapid restoration of AQP-4 channels (arrowed) by 5 h after injury.

FIG. 2 shows the effects of a neurokin-1 (NK1) antagonist on cerebral oedema as assessed by diffusion weighted imaging of rat brain 5 h following severe, diffuse traumatic brain injury. After a traumatic injury (left-hand panel), areas of hyperintensity reflect an increased ADC, which at this early time point is indicative of vasogenic edema. Treatment with the NK1 antagonist 30 min after the brain insult clearly reduces areas of hyperintensity within 4 h of administration (right-hand panel).

FIG. 3 shows the effects of a neurokin-1 (NK1) antagonist on intracranial pressure in the sheep model of diffuse traumatic brain injury. In sham (uninjured) sheep, ICP is between 10 and 15 mm Hg (open circles). After traumatic injury, in those animals that received only the saline vehicle (placebo) ICP is already increased to above 20 mm Hg as early as 30 min after the traumatic insult and continues to increase over time (open squares). The value of 20 mm Hg is considered problematic in human head injury, while 30 mm Hg is associated with brain herniations. In those animals that were administered an NK1 antagonist at 30 min after injury, there was an immediately decrease in ICP, with it returning to normal sham (control) levels over the ensuing 3 hours (closed squares).

FIG. 4 shows the effects of a neurokin-1 (NK1) antagonist on brain oxygen levels in the sheep model of diffuse traumatic brain injury. In sham (uninjured) sheep, brain oxygen levels are typically around 45 mmHg (open circles). After traumatic injury, in those animals that received only the saline vehicle (placebo) brain oxygen levels fall significantly, and decline to critically low levels within a few hours of the injury (open squares). In those animals that were administered an NK1 antagonist at 30 min after injury, there was an immediate improvement in brain oxygen status, such that by 4 hours after TBI, values have returned to normal (control) levels (closed squares).

FIG. 5 shows the relationship between intracranial pressure and brain oxygen levels in the sheep model of diffuse traumatic brain injury. Following injury, intracranial pressure and brain oxygen levels were monitored in parallel, and the values taken at the same time points were correlated. The results indicate that a linear relationship exists between these two variables when ICP lies between 10 and 25 mm Hg. Above an ICP of 25 mm Hg, the brain is clearly maximally oxygen deprived.

FIG. 6 shows the effects of the NK1 antagonist on ICP after trauma in the rat. Male Sprague Dawley rats were injured using a diffuse traumatic brain injury model, and then made hypoxic for a period of 15 minutes, before being ventilated on normal air. ICP was monitored using a Codman ICP pressure sensor for the ensuing 4 hours. After injury, ICP increased from a mean of 4.88±0.12 mm Hg to a mean of 9.23±0.28 mm Hg, an increase of 96%. Treatment with the NK1 antagonist, NAT, reduced ICP by a mean value 21%.

### GENERAL DESCRIPTION OF THE INVENTION

As described above, in one embodiment the present invention provides a method of reducing intracranial pressure in a subject, the method including administering to the subject an effective amount of a substance P receptor antagonist.

The subject in the various embodiments of the present invention is a human or animal subject.

In one embodiment, the method may be used to reduce intracranial pressure in a subject susceptible to raised intracranial pressure.

In an alternative embodiment, the method may be used to reduce intracranial pressure in a subject suffering from raised intracranial pressure.

It will be appreciated that the present invention may be used to reduce intracranial pressure generally, including reducing intracranial pressure when the pressure is in the normal range, and/or when intracranial pressure is elevated.

In this regard, normal intracranial pressure is generally considered to be a pressure in the range of 0 to 15 mm Hg, and thus a pressure above this range is considered to be raised intracranial pressure. However, there may be some conditions where a raised intracranial pressure is clinically significant at a pressure in the “normal” range, and the present invention may be used to reduce the intracranial pressure in that particular setting. Indirect and direct methods for measuring intracranial pressure are known in the art.

Raised intracranial pressure in the various embodiments of the present invention may be a consequence of a many diseases, conditions and states including stroke, acute brain injury, brain trauma, neurosurgical intervention, cranial haemorrhaging, cytotoxic and/or vasogenic oedema, peritumoural oedema, ischaemia, an infection of the central nervous system, neoplasia, liver failure, chronic obstructive lung disease, malaria, altitude sickness, and obstructive hydrocephalus.

The substance P receptor antagonist in the various embodiments of the present invention is an agent that directly or indirectly inhibits the binding of substance P to one of its receptors.

In this regard, substance P is an excitatory neurotransmitter and is a peptide having the structure RPKPEEFFGLM-NH₂. Substance P binds to a number of receptors including the NK1 receptor (neurokinin 1 receptor), the NK2 receptor and the NK3 receptor. Substance P antagonists inhibit the binding of substance P to one of its receptors. It will be appreciated that the term “substance P” includes within its scope various truncated forms or analogues of the peptide, for example as described in U.S. Pat. No. 4,481,139.

The identification of a substance as a substance P receptor antagonist may be determined by a method known in the art, for example as described U.S. Pat. No. 5,990,125.

For example, assays for NK1 activity may include ex vivo binding activity. In such assays the ability of the putative antagonist (test agent) to inhibit binding of labeled substance P to central and/or peripheral NK1 receptors provides an indication that such agents are potential NK1 antagonists. Methods of performing such binding assays are well known to those of skill in the art (see, e.g. Cascieri et al. (1995) Mol. Pharmacol., 47: 660-665).

Examples of substance P receptor antagonists are shown in Tables 1 to 3.

TABLE 1
NK1 Receptor Antagonists

| Chemical Code | Chemical Name |
|---|---|
| CGP49823 | (2R,4S)-2-benzyl-1-(3,5-dimethylbenzoyl)-N-[(4-quinolinyl)methyl]-4-piperineamine) dihydrochloride |
| CP-96,345 | 2S,3S)-cis-(2(diphenylmethyl)-N-[(2-methoxyphenyl)methyl]-1-azabicyclo[2.2.2]octan-3-amine |
| CP-99,994 | ((2S,3S)-cis-3-(2-methoxybenzylamino)-2-phenyl-piperidine)dihydrochloride |
| CP-122,721 | (+)-2S,3S)-3-(2-methoxy-5-trifluoromethoxybenzyl)amino-2-phenylpiperidine |
| FK 888 | (N2-[(4R)-4-hydroxy-1-(1-methyl-1H-indol-3-yl)carbonyl-L-propyl\-N-methyl-N-phenylmethyl-L-3-(2- |
| | naphthyl)-alaninamide |
| GR203040 | (2S,3S and 2R,3R)-2-methoxy-5-tetrazol-1-yl-benzyl-(2-phenyl-piperidin-3-yl)-amine |
| GR-205171 | 3-Piperidinamine,N-[[2-methoxy-5-[5-(trifluoromethyl)-1H-tetrazol-1yl]phenyl]methyl]-2-phenyl-, (2S-cis)- |
| GR 82334 | [D-Pro9,)spiro-gamma-lactam]Leu10,Trp11]physalaemin-(1-11) |
| GR 94800 | PhCO-Ala-Ala-DTrp-Phe-DPro-Pro-Nle-NH2 |
| HSP-117 | 3-Piperidinamine, N-[[2,3-dihydro-5-(1-methylethyl)-7-benzofuranyl]methyl)2-phenyl-, dihydrochloride, |
| | (2S-cis)- |
| L 703,606 | 1-Azabicycio[2.2.]octan-3-amine, 2-(diphenylmethyl)-N-[(2-idophenyl)methyl]-, (2S-cis)-, oxalate |
| L 732,138 | N-acetyl-L-tryptophan |
| L 733,060 | ((2S,S)-3-((3,5-bis(trifluoromethyl)phenyl)methyloxy)-2-phenyl piperidine |
| L 742,694 | (2-(S)-(3,5-bis(trifluromethyl)benzyloxy)-3-(S)-phenyl-4-(5-(3-oxo-1,2,4-triazolo)methylmorpholine |
| L 754,030 | 2-(R)-(1-(R)-3,5-bis(trifluoromethyl)phenylethoxy)-3-(S)-(4-fluoro)phenyl-4-(3-oxo-1,2,4-triazol-5- |
| | yl)methylmorpholine |
| L668,169 | L-Phenylalanine, N-[2-[3-[[N-[2-(3-[(N-[2-[3-amino-2-oxo-1-pyrrolidinyl)-4-methyl-1-oxopentlyl]-L- |
| | methionyl-L-glutaminyl-D-tryplophyl-N-methyl-L-phenylalanyl]amino]-2-oxo-1-pyrrolidinyl]-4-methyl-1- |
| | oxopentyl]-L-methionyl-L-glutaminyl-D-tryptophyl-N-methyl-,cyclic (8−>1)-peptide, [3R-[1[S*[R*(S*)]], |
| | 3R*]]- |
| LY 303241 | 1-Piperazineacetamide, N-[2-[acetyl[(2-methoxyphenyl)methyl]amino]-1-(1H-indol-3-ylmethyl)(ethyl]-4- |
| | phenyl-, (R)- |
| LY 303870 | (R)-1-[N-(2-methoxybenzyl)acetylamino]-3-(1H-indol-3-yl)-2-[N-(2-(4-(piperidinyl)piperidin-1- |
| | yl)acetyl)amino]propane |
| LY 306740 | 1-Piperazineacetamide, N-[2-′acetyl[(2-methoxypehenyl)methyl]amino]-1-(1H-indol-3-ylmethyl)ethyl]-4- |
| | cyclohexyl-, (R)- |
| MEN 11149 | 2-(2-naphthyl)-1-N[(1R,2S)-2-N-[1(H)indol-3-ylcarbonyl]aminocyclohexanecarbonyl]-1-[N′-ethyl-N′-(4- |
| | methylphenylacetyl)] diaminoethane |
| MK-869 | 3H-1,2,4-Triazol-3-one, 5-[[2-[1-[3,5-bis(trifuoromethyl)phenyl]ethoxy]-3-(4-fluorophenyl)-4- |
| | morpholinyl]methyl]-1,2-dihydro-,[2R-[2a(R*),3a]]- |
| PD 154075 | (2-benzofuran)-CH2OCO]-(R)-alpha-MeTrp-(S)-NHCH(CH3)Ph |
| R-544 | Ac-Thr-D-Trp(FOR)-Phe-N-MeBzl |
| RP-67580 | (3aR,7aR)-7,7-diphenyl-2[1-imino-2(2-methoxyphenyl)-(ethyl]+++perhydroisoindol-4-one hydrochloride |
| RPR 100893 | (3aS,4S,7aS)-7,7-diphenyl-4-(2-methoxyphenyl)-2-[(S)-2-(2-methoxyphenyl)proprionyl]perhydroisoindol- |
| | 4-ol |
| Spendide | Tyr-D-Phe-Phe-D-His-Leu-Met-NH2 |
| Spantide II | D-NicLys1,3-Pal3,D-C12Phe5,Asn6,D-Trp7.0,Nle11-substance P |
| Spantide III | L-Norleucinamide, N6-(3-pyridinylcarbonyl)-D-lysyl-L-prolyl-3-(3-pyridinyl)-L-alanyl-L-prolyl-3,4- |
| | dichioro-D-phenylalanyl-L-asparaginyl-D-tryptophyl-L-phenylalanyl-3-(3-pyridinyl)-D-alanyl-L-leucyl- |
| SR140333 | (S)-1-[2-[3-(3,4-dichlorphenyl)-1 (3-isopropoxyphenylacetyl) piperidin-3-yl] ethyl]-4-phenyl-1 |
| | azaniabicyclo [2.2.2]octane |
| WIN-41,708 | (17beta-hydroxy-17alpha-ethynyl-5alpha-androstano[3,2-b]pyrimido[1,2-a]benzimidazole |
| WIN-62,577 | 1H-Benzimidazo[2,1-b]cyclopenta[5,6]naphtha[1,2-g]quinazolin-1-ol, 1-ethynyl- |
| | 2,3,3a,3b,4,5,15,15a,15b,16,17,17a-dodeachydro-15a,17a-dimethyl-, (1R,3aS,3bR,15aR,15bS,17aS)- |

TABLE 2
NK2 Receptor Antagonists

| Chemical Code | Chemical Name |
|---|---|
| SR-48,968 | (S)-N-methyl-N[4-(4-acetylamino-4-[phenylpiperidino)-2-(3,4-dichlorophenyl)-butyl]benzamide |
| L-659,877 | Cyclo[Gin,Trp,Phe,Gly,Leu,Met] |
| MEN 10627 | Cyclo(Met-Asp-Trp-Phe-Dap-Leu)cyclo(2beta-5beta) |
| SR 144190 | (R)-3-(1-[2-(4-benzoyl-2-(3,4-difluorophenyl)-morpholin-2-yl)-ethyl]-4-phenylpiperidin-4-yl)-1- |
| | dimethylurea |
| GR 94800 | PhCO-Ala-Ala-D-Trp-Phe-D-Pro-Pro-Nle-NH2 |

TABLE 3
NK3 Receptor Antagonists

| Chemical Code | Chemical Name |
|---|---|
| SR-142,801 | (S)-(N)-(1-(3-(1-benzoyl-3-(3,4-dichlorophenyl)piperidin-3-yl)propyl)-4-phenylpiperidin-4-yl)-N-methyl |
| | acetaide |
| R820 | 3-Indolylcarbonyl-Hyp-Phg-N(Me)-Bzl |
| R486 | H-Asp-Ser-Phe-Trp-beta-Ala-Leu-Met-NH2 |
| SB 222200 | (S)-(−)-N-(a-ethylbenzyl)-3-methyl-2-phenylquinoline-4-carboximide |
| L 758,298 | Phosphonic acid, [3-[[2-[1-[3,5-bis(trfluoromethyl)phenyl]ethoxy]-3-(4-fluorophenyl)-4- |
| | morpholinyl]methyl]-2,5-dihydro-4oxo-1H-1,2,4-triazol-1-yl]-, [2R-[2a(R*),3a]]- |
| NK-608 | (2R,4S)-N-[1-[3,5-bis(trifluormethyl)-benzoyl)-2-(4-chloro-benzyl)-4-piperidinyl]-quinoline-4-carboxamide |

Other examples of substance P receptor antagonists include those described in U.S. Pat. Nos. 4,481,139 and 5,977,104.

Examples of NK1 receptor antagonists include those described in U.S. Pat. No. 5,990,125 and U.S. Pat. No. 7,122,677.

Tachykinin antagonists (as described in U.S. Pat. No. 4,981,744) may also be used as substance P antagonists. Other examples of substance P receptor antagonists include piperidine and morpholine derivatives (as described in U.S. Pat. No. 4,985,896), piperazino (as described in U.S. Pat. No. 5,981,52), piperidinyl compounds as NK1 or NK2 antagonists (as described in U.S. Pat. No. 5,998,444), N-benzyl-4-tolylnicotinamides and related compounds as NK1 receptor antagonists (as described in European patent application EP-A-1035115), phenyl and pyridinyl derivatives as NK1 receptor antagonists (as described in international patent application WO 0050398), and 3-phenylpyridines, biphenyl derivatives, 5-phenyl-pyrimidine derivatives and 4-phenyl-pyrimidine derivatives (as described in international patent applications WO 00/50401, WO 00/53572, WO 00/73278 and WO 00/73279).

In one embodiment, the substance P receptor antagonist is selected from one of the group consisting of a NK1 receptor antagonist, a NK2 receptor antagonist, or a NK3 receptor antagonist.

In one embodiment, the NK1 receptor antagonist is selected from one or more of the group consisting of CGP49823, CP-96,345, CP99,994, CP-122,721, FK88, GR203040, GR205171, GR82334, GR94800, HSP-117, L-703,606 oxalate, L-732,138 (N-acetyl-L-tryptophan), L-733060, L-742,694, L-745,030, L-668,169, LY-303241, LY-303870, LY306740, MEN-11149, MK-869, PD-154075, R-544, RP-67580, RPR100893, Sendide, Spantide II, Spantide III, SR140333, WIN-41,7098, WIN-62,577.

In another embodiment, the NK2 receptor antagonist is selected from one or more of the group consisting of SR-48968, L-659877, GR103537, MGN-10627, SR144190 and GR94800.

In another embodiment, the NK3 receptor antagonist is selected from one or more of the group consisting of SR-143,801, R820, R486, SB222200, L758,298 and NKP608.

The present invention may also be used in the preparation of a medicament for reducing intracranial pressure in a subject.

Accordingly, in another embodiment the present invention provides use of a substance P receptor antagonist in the preparation of a medicament for reducing intracranial pressure in a subject.

The present invention may also be used to prevent and/or treat raised intracranial pressure in a subject.

Accordingly, in another embodiment the present invention provides a method of preventing and/or treating raised intracranial pressure in a subject, the method including administering to the subject an effective amount of a substance P receptor antagonist.

In one specific embodiment, the present invention may be used to treat raised intracranial pressure in a subject suffering from this condition.

The present invention may also be used in the preparation of a medicament for preventing and/or treating raised intracranial pressure in a subject.

Accordingly, in another embodiment the present invention provides use of a substance P receptor antagonist in the preparation of a medicament for preventing and/or treating raised intracranial pressure in a subject.

The present invention may also be used to improve blood flow in a subject suffering from raised intracranial pressure. In this regard, one of the consequences of a rise in intracranial pressure is a reduction in blood perfusion of the brain. Thus, administration of a substance P receptor antagonist may be used to restore blood perfusion of the brain, and thereby prevent and/or ameliorate the cycle of events that occur once intracranial pressure begins to rise.

Accordingly, in another embodiment the present invention provides a method of improving blood flow to the brain in a subject suffering from raised intracranial pressure, the method including administering to the subject an effective amount of a substance P receptor antagonist.

Methods for determining the extent of blood flow into the brain are known in the art.

The present invention may also be used in the preparation of a medicament for improving blood flow to the brain in a subject suffering from raised intracranial pressure.

Accordingly, in another embodiment the present invention provides use of a substance P receptor antagonist in the preparation of a medicament for improving blood flow to the brain in a subject suffering from raised intracranial pressure.

The present invention may also be used to improve oxygen delivery and/or oxygenation of brain tissue in a subject suffering from raised intracranial pressure. In this regard, one of the consequences of a rise in intracranial pressure is a reduction in brain tissue oxygenation. Thus, administration of a substance P receptor antagonist may be used to restore oxygenation of the brain tissue, and thereby prevent and/or ameliorate the cycle of events that occur once intracranial pressure begins to rise.

Accordingly, in another embodiment the present invention provides a method of improving oxygen delivery to the brain and/or improving oxygenation of the brain in a subject suffering from raised intracranial pressure, the method including administering to the subject an effective amount of a substance P receptor antagonist.

Methods for determining the extent of oxygen delivery to the brain, and for determining the extent of brain tissue oxygenation, are known in the art.

The present invention may also be used in the preparation of a medicament for improving oxygen delivery or oxygenation of the brain in a subject suffering from raised intracranial pressure.

Accordingly, in another embodiment the present invention provide use of a substance P receptor antagonist in the preparation of a medicament for improving oxygen delivery to the brain and/or improving oxygenation of the brain in a subject suffering from raised intracranial pressure.

The present invention may also be used to improve movement of water from the brain into the circulation.

Accordingly, in another embodiment the present invention provides a method of improving movement of water from the brain of a subject into the circulation, the method including administering to the subject an effective amount of a substance P receptor antagonist.

Methods for determining the extent of water flow from the brain into the circulation are known in the art.

The present invention may also be used in the preparation of a medicament for improving movement of water from the brain of a subject into the circulation.

Accordingly, in another embodiment the present invention provides use of a substance P receptor antagonist in the preparation of a medicament for improving movement of water from the brain of a subject into the circulation.

The present invention may also be used to reduce the risk of mortality or morbidity in a subjection susceptible to, or suffering from, raised intracranial pressure. In this regard, one of the final consequences of a rise in intracranial pressure is death or neurological damage. Thus, administration of a substance P receptor antagonist may be used to reduce the risk of mortality or morbidity associated with raised intracranial pressure.

In this regard, the term “risk” is to be understood to be the probability that the subject would suffer an event in the absence of intervention. Generally, the risk is determined by measuring the rate of the event occurring in a population of subjects not subject to intervention, as compared to the rate of the event occurring in a population of subject undergoing intervention.

Accordingly, in another embodiment the present invention provides a method of reducing the risk of mortality or morbidity in a subject susceptible to, or suffering from, raised intracranial pressure, the method including administering to the subject an effective amount of a substance P receptor antagonist.

The present invention may also be used in the preparation of a medicament for reducing the risk of mortality or morbidity in a subject susceptible to, or suffering from, raised intracranial pressure.

Accordingly, in another embodiment the present invention provides use of a substance P receptor antagonist in the preparation of a medicament for reducing the risk of mortality or morbidity in a subject susceptible to, or suffering from, raised intracranial pressure.

The present invention may also be used to reduce the risk of a neurological complication due to raised intracranial pressure in a subject. In this regard, one of the consequences of a rise in intracranial pressure is a neurological complication. Thus, administration of a substance P receptor antagonist may be used to reduce the risk of a neurological complication associated with raised intracranial pressure

Accordingly, in another embodiment the present invention provides a method of reducing the risk of a neurological complication due to raised intracranial pressure in a subject, the method including administering to the subject an effective amount of a substance P receptor antagonist.

The present invention may also be used in the preparation of a medicament for reducing the risk of a neurological complication due to raised intracranial pressure.

Accordingly, in another embodiment the present invention provides use of a substance P receptor antagonist in the preparation of a medicament for reducing the risk of a neurological complication due to raised intracranial pressure in a subject.

The present invention may also be used to improve the prognosis or outcome of a subject suffering from raised intracranial pressure.

Accordingly, in another embodiment the present invention provides a method of improving the prognosis or outcome of a subject suffering from raised intracranial pressure, the method including administering to the subject an effective amount of a substance P receptor antagonist.

In one embodiment, the improvement in prognosis or outcome is an improvement in a neurological prognosis or outcome.

The present invention may also be used in the preparation of a medicament for improving the prognosis or outcome of a subject suffering from raised intracranial pressure.

Accordingly, in another embodiment the present invention also provides use of a substance P receptor antagonist in the preparation of a medicament for improving the prognosis or outcome of a subject suffering from raised intracranial pressure.

The administration of the substance P receptor antagonist in the various embodiments of the present invention may further include administration of one or more agents that may be used as adjunctive therapy for intrancranial pressure or one of its consequences. For example, an osmotic diuretic, such as mannitol, may be used in combination with a substance P receptor antagonist.

Accordingly, in another embodiment the present invention provides a pharmaceutical composition including a substance P receptor antagonist and an osmotic diuretic.

In this regard, the one or more agents and the substance P receptor antagonist in the various embodiments of the present invention may be administered delivered simultaneously or sequentially.

In this regard, co-administration generally means that the actives are present in the subject during a specified time interval. Typically, if a second agent is administered within the half-life of the first agent, the two agents are considered co-administered.

Thus, the present invention may also be used for a combination product including a substance P receptor antagonist and another agent.

In another embodiment, the present invention provides a combination product including the following components:

a substance P receptor antagonist; and

an osmotic diuretic;

wherein the components are provided in a form for co-administration to a subject or in a form for separate administration to a subject.

The administration of the substance P receptor antagonist in the various embodiments of the present invention may include the administration of a pharmaceutical composition including the substance P receptor antagonist. In this case, the substance P receptor antagonist may be prepared into a suitable pharmaceutical composition.

Accordingly, in another embodiment the present invention provides a pharmaceutical composition including an effective amount of a substance P receptor antagonist. Such pharmaceutical compositions may be used to reduce intracranial pressure, and/or prevent and/or treat one of its consequences.

A suitable dosage of the substance P receptor antagonist for reducing intracranial pressure (and/or for preventing and/or treating one of its consequences) may be selected. Generally, the dosage of the substance P receptor antagonist administered to a subject in the various embodiments of the present is in the range from 0.1 mg/kg to 100 mg/kg. Typically, the dosage is in the range from 0.25 mg/kg to 25 mg/kg. For example, a suitable dose of N-acetyl-tryptophan is 2.5 mg/kg.

Generally, the dosage of the substance P receptor antagonist in the pharmaceutical composition may be in the range from 10-5,000 mg per subject, and typically will be in the range of 50-2,000 mg per subject.

Suitable dosages are generally as described in U.S. Pat. No. 4,990,125 and U.S. Pat. No. 5,977,104.

Methods for the preparation of pharmaceutical compositions are known in the art, for example as described in Remington's Pharmaceutical Sciences, 18th ed., 1990, Mack Publishing Co., Easton, Pa. and U.S. Pharmacopeia: National Formulary, 1984, Mack Publishing Company, Easton, Pa.

Examples of formulations of substance P receptor antagonists are generally as described in U.S. Pat. No. 5,990,125, and US patent application 20070032491.

The substance P receptor antagonist may be delivered directly or indirectly to the desired site of action. For example, direct delivery may be achieved by injection into the brain or cerebral vasculature.

Alternatively, in the various embodiments of the present invention, the substance P receptor antagonist may be delivered indirectly to the desired site of action in a subject by way, for example by way of systemic administration to the subject.

The substance P receptor antagonist may be delivered in a form and at a concentration suitable to allow the agent to reach the desired site of action and have the desired effect, as previously discussed herein.

The administration of the substance P receptor antagonist in the various embodiments of the present invention may utilise a suitable administration regime to produce the desired effect.

For example, the substance P receptor antagonist may be administered to a subject before a rise in intracranial pressure occurs, and/or after a rise in intracranial pressure has occurred. Thus the present invention includes prophylactic administration of the substance P receptor to a subject susceptible to raised intracranial pressure, and/or therapeutic administration of the substance P receptor antagonist to a subject suffering from raised intracranial pressure.

The delivery of the substance P receptor antagonist may be by any suitable means, such as administered by injection, orally, parenterally, topically, and therefore transit time of the agent must be taken into account.

The substance P receptor antagonist may be formulated into a pharmaceutical composition for administration to a subject, and as such the composition may be packaged in a suitably sterilized container such as an ampoule, bottle, or vial, either in multi-dose or in unit dosage forms. Containers will generally be hermetically sealed. Methods are known in the art for the packaging of components for pharmaceutical administration.

The effective amount of the substance P receptor antagonist to be administered to the subject in the various embodiments of the present invention is not particularly limited, so long as it is within such an amount and in such a form that generally exhibits a useful or therapeutic effect. The term “therapeutically effective amount” is the quantity which, when administered to a subject in need of treatment, improves the prognosis and/or state of the subject. The amount to be administered to a subject will depend on the particular nature of the clinical presentation, the mode of administration, and the characteristics of the subject, such as general health, other diseases, age, sex, genotype, body weight and tolerance to drugs. A person skilled in the art will be able to determine appropriate dosages depending on these and other factors.

As discussed previously herein, administration and delivery of the compositions according to the invention may be, for example, by intravenous, intraperitoneal, subcutaneous, intramuscular, oral, or topical routes, or by direct injection. The mode and route of administration in most cases will depend on the nature of the clinical presentation of the subject being treated.

The dosage form, frequency and amount of dose will depend on the mode and route of administration.

The administration of the substance P receptor antagonist may also include the use of one or more pharmaceutically acceptable additives, including pharmaceutically acceptable salts, amino acids, polypeptides, polymers, solvents, buffers, excipients, preservatives and bulking agents, taking into consideration the particular physical, microbiological and chemical characteristics of the agent to be administered.

For example, the substance P receptor antagonist can be prepared into a variety of pharmaceutically acceptable compositions in the form of, for example, an aqueous solution, an oily preparation, a fatty emulsion, an emulsion, a lyophilised powder for reconstitution, etc. and can be administered as a sterile and pyrogen free intramuscular or subcutaneous injection or as injection to an organ, or as an embedded preparation or as a transmucosal preparation through nasal cavity, rectum, uterus, vagina, lung, etc. The composition may be administered in the form of oral preparations (for example solid preparations such as tablets, caplets, capsules, granules or powders; liquid preparations such as syrup, emulsions, dispersions or suspensions).

Compositions containing the substance P receptor antagonist may also contain one or more pharmaceutically acceptable preservatives, buffering agents, diluents, stabilisers, chelating agents, viscosity enhancing agents, dispersing agents, pH controllers, or isotonic agents. These excipients are well known to those skilled in the art.

Examples of suitable preservatives are benzoic acid esters of para-hydroxybenzoic acid, propylene glycol, phenols, phenylethyl alchohol or benzyl alcohol. Examples of suitable buffers are sodium phosphate salts, citric acid, tartaric acid and the like. Examples of suitable stabilisers are, antioxidants such as alpha-tocopherol acetate, alpha-thioglycerin, sodium metabisulphite, ascorbic acid, acetylcysteine, 8-hydroxyquinoline, chelating agents such as disodium edetate. Examples of suitable viscosity enhancing agents, suspending or dispersing agents are substituted cellulose ethers, substituted cellulose esters, polyvinyl alchohol, polyvinylpyrrolidone, polyethylene glcols, carbomer, polyoxypropylene glycols, sorbitan monooleate, sorbitan sesquioleate, polyoxyethylene hydrogenated castor oil 60.

Examples of suitable pH controllers include hydrochloric acid, sodium hydroxide and the like. Examples of suitable isotonic agents are glucose, D-sorbitol or D-mannitol, sodium chloride.

The administration of the substance P receptor antagonist in the various embodiments of the present invention may also be in the form of a composition containing a pharmaceutically acceptable carrier, diluent, excipient, suspending agent, lubricating agent, adjuvant, vehicle, delivery system, emulsifier, disintegrant, absorbent, preservative, surfactant, colorant, glidant, anti-adherant, binder, flavorant or sweetener, taking into account the physical, chemical and microbiological properties of the agent being administered.

For these purposes, the composition may be administered orally, parenterally, by inhalation spray, adsorption, absorption, topically, rectally, nasally, mucosally, transdermally, bucally, vaginally, intraventricularly, via an implanted reservoir in dosage formulations containing conventional non-toxic pharmaceutically-acceptable carriers, or by any other convenient dosage form. The term parenteral as used herein includes subcutaneous, intravenous, intramuscular, intraperitoneal, intrathecal, intraventricular, intrasternal, and intracranial injection or infusion techniques.

When administered parenterally, the compositions will normally be in a unit dosage, sterile, pyrogen free injectable form (solution, suspension or emulsion, which may have been reconstituted prior to use) which is preferably isotonic with the blood of the recipient with a pharmaceutically acceptable carrier. Examples of such sterile injectable forms are sterile injectable aqueous or oleaginous suspensions. These suspensions may be formulated according to techniques known in the art using suitable vehicles, dispersing or wetting agents and suspending agents. The sterile injectable forms may also be sterile injectable solutions or suspensions in non-toxic parenterally acceptable diluents or solvents, for example, as solutions in 1,3-butanediol. Among the pharmaceutically acceptable vehicles and solvents that may be employed are water, ethanol, glycerol, saline, Ringer's solution, dextrose solution, isotonic sodium chloride solution, and Hanks' solution. In addition, sterile, fixed oils are conventionally employed as solvents or suspending mediums. For this purpose, any bland fixed oil may be employed including synthetic mono- or di-glycerides, corn, cottonseed, peanut, and sesame oil. Fatty acids such as ethyl oleate, isopropyl myristate, and oleic acid and its glyceride derivatives, including olive oil and castor oil, especially in their polyoxyethylated versions, are useful in the preparation of injectables. These oil solutions or suspensions may also contain long-chain alcohol diluents or dispersants.

The carrier may contain minor amounts of additives, such as substances that enhance solubility, isotonicity, and chemical stability, for example anti-oxidants, buffers and preservatives.

In addition, the compositions may be in a form to be reconstituted prior to administration. Examples include lyophilisation, spray drying and the like to produce a suitable solid form for reconstitution with a pharmaceutically acceptable solvent prior to administration.

Compositions may include one or more buffers, bulking agents, isotonic agents and cryoprotectants and lyoprotectants. Examples of excipients include, phosphate salts, citric acid, non-reducing such as sucrose or trehalose, polyhydroxy alcohols, amino acids, methylamines, and lyotropic salts are preferred to the reducing sugars such as maltose or lactose.

When administered orally, the substance P receptor antagonist will usually be formulated into unit dosage forms such as tablets, caplets, cachets, powder, granules, beads, chewable lozenges, capsules, liquids, aqueous suspensions or solutions, or similar dosage forms, using conventional equipment and techniques known in the art. Such formulations typically include a solid, semisolid, or liquid carrier. Exemplary carriers include excipients such as lactose, dextrose, sucrose, sorbitol, mannitol, starches, gum acacia, calcium phosphate, mineral oil, cocoa butter, oil of theobroma, alginates, tragacanth, gelatin, syrup, substituted cellulose ethers, polyoxyethylene sorbitan monolaurate, methyl hydroxybenzoate, propyl hydroxybenzoate, talc, magnesium stearate, and the like.

A tablet may be made by compressing or molding the agent optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispersing agent. Moulded tablets may be made by moulding in a suitable machine, a mixture of the powdered active ingredient and a suitable carrier moistened with an inert liquid diluent.

The administration of the substance P receptor antagonist may also utilize controlled release technology.

The substance P receptor antagonist may also be administered as a sustained-release pharmaceutical composition. To further increase the sustained release effect, the agent may be formulated with additional components such as vegetable oil (for example soybean oil, sesame oil, camellia oil, castor oil, peanut oil, rape seed oil); middle fatty acid triglycerides; fatty acid esters such as ethyl oleate; polysiloxane derivatives; alternatively, water-soluble high molecular weight compounds such as hyaluronic acid or salts thereof, carboxymethylcellulose sodium hydroxypropylcellulose ether, collagen polyethylene glycol polyethylene oxide, hydroxypropylmethylcellulo semethylcellulo se, polyvinyl alcohol, polyvinylpyrrolidone.

Alternatively, the substance P receptor antagonist may be incorporated into a hydrophobic polymer matrix for controlled release over a period of days. The agent may then be moulded into a solid implant, or externally applied patch, suitable for providing efficacious concentrations of the agents over a prolonged period of time without the need for frequent re-dosing. Such controlled release films are well known to the art. Other examples of polymers commonly employed for this purpose that may be used include nondegradable ethylene-vinyl acetate copolymer a degradable lactic acid-glycolic acid copolymers, which may be used externally or internally. Certain hydrogels such as poly(hydroxyethylmethacrylate) or poly(vinylalcohol) also may be useful, but for shorter release cycles than the other polymer release systems, such as those mentioned above.

The carrier may also be a solid biodegradable polymer or mixture of biodegradable polymers with appropriate time-release characteristics and release kinetics. The agent may then be moulded into a solid implant suitable for providing efficacious concentrations of the agents over a prolonged period of time without the need for frequent re-dosing. The agent can be incorporated into the biodegradable polymer or polymer mixture in any suitable manner known to one of ordinary skill in the art and may form a homogeneous matrix with the biodegradable polymer, or may be encapsulated in some way within the polymer, or may be moulded into a solid implant.

It will be appreciated that other forms of administration of polypeptide based agents are also contemplated, including the use of nucleic acid encoding the polypeptides for delivering the agents. For example, therapeutic delivery of biolomolecules is generally as described in Bladon, C. (2002) “Pharmaceutical Chemistry: Therapeutic Aspects of Biomolecules” John Wiley & Sons Ltd.

### DESCRIPTION OF SPECIFIC EMBODIMENTS

Reference will now be made to experiments that embody the above general principles of the present invention. However, it is to be understood that the following description is not to limit the generality of the above description.

### Example 1

### Aquaporin-4 Expression is Reduced after Traumatic Brain Injury

In terms of trying to lower intracranial pressure, osmotic diuretics, like mannitol, are routinely used, although it is recognised that their efficacy is very limited. These agents are pharmacologically inert substances that act by increasing the colloid osmotic pressure of the blood, providing a driving force for the reabsorption of water from a tissue back into the circulation. However, their ability to draw water out of the brain in a situation where there is raised intracranial pressure is questionable.

This suggested to us that, following an insult, a change must occur in the cerebral vasculature that limits the ability of water to move back out of the brain. We decided to study the water channels associated with the cerebral vasculature, and in particular aquaporin 4 (AQP-4), in order to see whether there were any changes in the expression of these channels. For these studies, a rat model was used, and raised ICP was induced by diffuse traumatic brain injury.

In this model, a traumatic brain injury was induced using the impact-acceleration model. Briefly, the impact-acceleration device delivers an accelerating blow centrally on to the dorsal surface of the skull of halothane anesthetized male Sprague-Dawley rats (380-420 g). The point of impact was protected with a stainless steel disc to prevent skull fracture, while the head was subsequently decelerated using either a foam or gel filled cushion. For severe injury, the 450 g brass weight is dropped from a height of 2 metres. The impact results in extensive, diffuse neuropathological damage throughout the brain with associated neurological deficits.

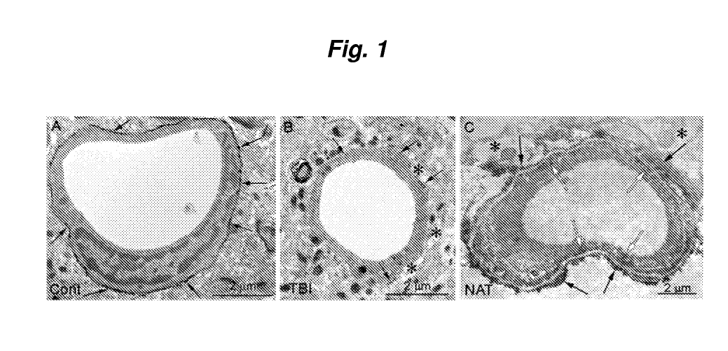

Immunohistochemical labelling was used to investigate the expression of aquaporin-4 (FIG. 1). Briefly, electron microscopy of AQP-4 used a rabbit polyclonal antibody (Alpha Diagnostic International, Inc., San Antonio) as the primary antibody, with immunolabelling completed using biotinylated secondary antibody (goat anti-rabbit IgG) and streptavidin-peroxidase complex (Rockland, Gilbertsville, Pa.). Rectangles of tissue were then cut from immunolabelled vibratome sections, osmicated and processed in resin, and ultrathin sections examined with the electron microscope for the detection of peroxidase reaction product.

AQP-4 expression was clearly observed in control animals (FIG. 1, left-hand panel). However, following injury, there is a very significant and unexpected reduction in the number of water channels associated with the vasculature (FIG. 1, centre panel).

We surmised that this finding would explain the common observation that osmotic diuretics are not effective in drawing water out of the brain. Whilst the diuretics may establish an osmotic gradient that should drive water reabsorption, if the water channels are lacking from the vasculature, then there is no route available to enable the water to pass back into the circulation.

### Example 2

### Administration of N-Acetyl-L-Tryptophan after Traumatic Brain Injury Induces expression of aquaporin 4

We hypothesised that substance P may play a role in bringing about these changes in vascular function, and therefore if we were to inhibit the action of substance P, using an NK1 receptor antagonist for example, we may be able to reverse these changes.

A number of commercially synthesised substance P receptor antagonists are currently available from standard scientific chemical suppliers. We chose to use the NK1 receptor antagonist N-acetyl-L-tryptophan.

N-acetyl-tryptophan was dissolved in sterile, normal saline, and administered by intravenous injection at 30 min after induction of injury. Administration of N-acetyl-L-tryptophan after injury was found to cause a rise in the expression of aquaporin-4 to, or indeed above, pre-injury levels (FIG. 1, right-hand panel). The optimal dose, based on the ability of the drug to attenuate blood brain barrier permeability after injury, was 2.5 mg/kg.

### Example 3

### Administration of N-Acetyl Tryptophan Facilitates Movement of Water from Brain Tissue Into the Circulation in a Rat Model of Traumatic Brain Injury

The changes in aquaporin-4 expression, as well the ability of a substance P receptor antagonist to restore normal expression levels, suggested that these agents may be enable the movement of water out of the brain, which would help to reduce intracranial pressure.

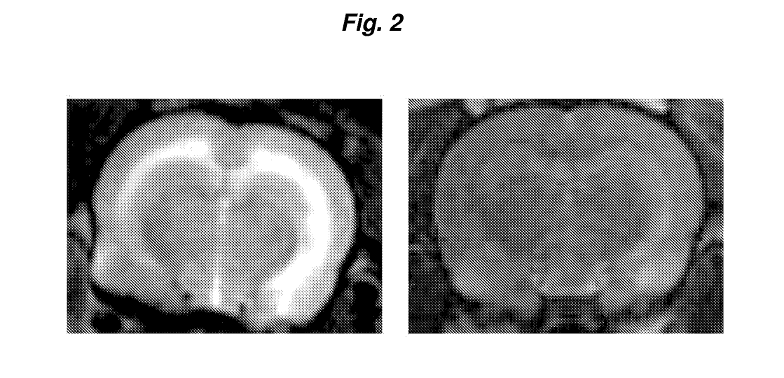

In order to observe whether a substance P receptor antagonist could facilitate the movement of water out of the brain, we carried out nuclear magnetic resonance spectroscopy (NMR) studies using a rat model of traumatic brain injury. Diffusion-weighted NMR images were generated following trauma (FIG. 2, left-hand panel).

Following injury, a number of “brighter” areas are clearly visible within the brain. These “bright” areas correlate with regions where extracellular water is gathering within the brain.

Following post-injury administration of a N-acetyl-L-tryptophan at a dose of 10⁻⁵ mol/kg, there is no indication of any water gathering in these areas (FIG. 2, left-hand panel), suggesting that the NK1 receptor antagonist has facilitated the movement of that water back into the circulation.

### Example 4

### Administration of N-Acetyl Tryptophan Reduces Intracranial Pressure in a Sheep Model Of Traumatic Brain Injury

In order to examine the potential clinical application of these findings, we conducted further studies using a large animal (sheep) model which mimics the brain trauma normally associated with motor vehicle accidents. The use of the large animal model enabled the direct measurement of the clinical parameters that are central to the management and prognosis of human patients.

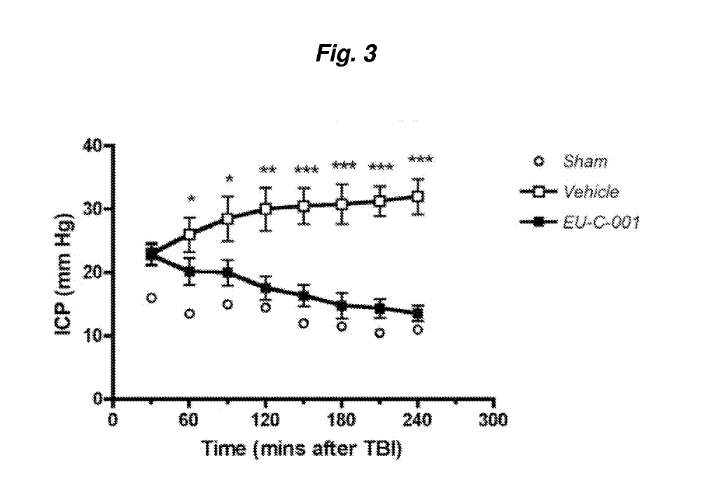

Following traumatic injury, intracranial pressure was measured in the sheep using a standard neurosurgical “bolt” and pressure transducer (FIG. 3).

Briefly, 2-year-old male Merino sheep were impacted in the left temporal region by a humane stunner. Sheep were continuously anesthetized and ventilated using 2.5% isoflurane in oxygen (4 L/min). Animals were then placed into a prone sphinx position, and restrained to the table, leaving the neck and head mobile relative to the body. Impact injury was induced at the midpoint between the left supraorbital process and the left external auditory meatus using a Captive humane bolt stunner armed with a number 7 red charge (model KML, Karl Schermer & Co., Germany). The injury produced by this method has been fully characterised, and mimics that seen in human TBI victims. (Lewis, S. B., Finnie, J. W., Blumbergs, P. C., Scott, G., Manavis, J. Brown, C., Reilly, P. L., Jones, N. R., McLean, A. J. (1996) A head impact model of early axonal injury in the sheep. J Neurotrauma 13, 505-514.)

After injury, animals were stabilised and the heads restrained to the operating table to facilitate insertion of intracranial pressure (ICP) and brain tissue oxygenation (PbtO2) probes between 15 and 30 min after trauma. Following exposure of the skull, a 5.8 mm burr hole was performed at a point 4 cm lateral to the sagittal midline on the ipsilateral side, the dura matter opened and a calibrated Codman Microsensor ICP transducer inserted such that the tip of the sensor was 1.5 cm into the parenchyma of the left parietal lobe. The probe was attached to a Codman ICP Express monitoring system (Codman and Shurtleff Inc., USA) for digital recording.

In the sheep, as in humans, normal intracranial pressure lies between 0 and 15 mm Hg. This can be evidenced by the open circles (sham). Following injury there is a rise in intracranial pressure, and in those animals subject to standard clinical supportive therapy (vehicle), the intracranial pressure continues to rise. When the ICP reached 30 mm Hg, clinical signs of brain herniation were observed. However, for those animals that were administered a NK receptor antagonist, the ICP began to fall after administration of the drug, and was within normal limits within 3-4 hours. Although individual variation was observed, the ICP always fell in those animals receiving the NK1 receptor antagonist, whilst it always rose in those not receiving the drug therapy.

### Example 5

### Administration of N-Acetyl Tryptophan Restores Oxygen Levels to Normal Following Traumatic Brain Injury

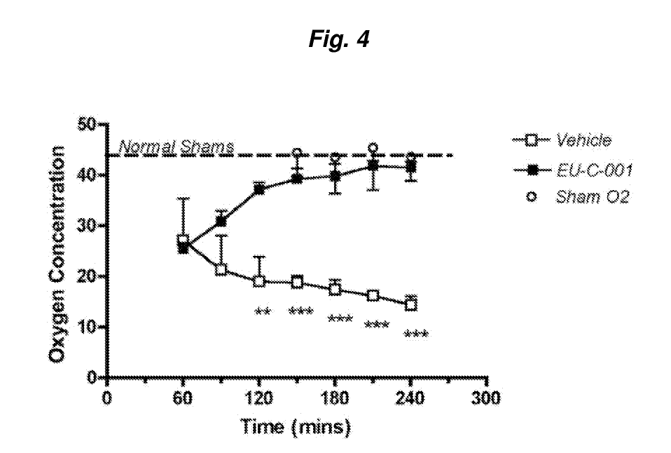

As indicated above, one of the main problems with a rise in intracranial pressure is that it impacts on blood flow and oxygen delivery to the brain. Current clinical evidence suggests that it is the fall in brain oxygen levels which is the primary indicator of patient outcome. Therefore, in addition to monitoring ICP, brain tissue oxygenation was also measured using a clinical oxygen probe (FIG. 4).

The injury was carried out in sheep as previously described. In addition to the burr hole for ICP monitoring, a second burr hole 1 cm lateral to the sagittal midline and over the left fronto-parietal suture allowed insertion of the distal end of a LICOX® PbtO2 probe to a depth of 2 cm. The probe was attached to a LICOX® brain tissue oxygen monitoring system (Integra, USA) for digital recording. After insertion of the probes, both burr holes were sealed using bone wax. ICP and PbtO2 was recorded every 30 min for a period of 4 hours.

Normal tissue oxygen concentration in the brain is around 44 mmHg, as observed in sham controls. Following injury there is a significant fall in tissue oxygen levels, and they continue to fall in those animals that received the standard clinical supportive therapy (vehicle). However, in those animals that received NK1 receptor antagonist after injury, the oxygen levels start to rapidly recover, and are back to normal limits within 3-4 hours. This indicates that there is a direct correlation between the impact of raised intracranial pressure and brain oxygen levels and, moreover, that NK1 receptor antagonists are capable of significantly reducing raised intracranial pressure, and thereby restoring normal blood and oxygen supply to the brain.

### Example 6

### Correlation Between Raised ICP and Oxygen Deprivation in the Brain

As indicated previously, raised intracranial pressure has a number of significant implications on the integrity of brain function. One of the most significant of these is its impact on cerebral blood flow, and the subsequent delivery of oxygen to the brain.

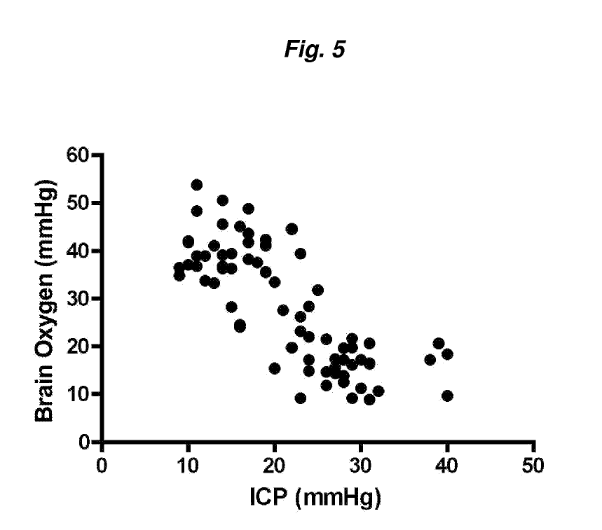

Using the sheep model of TBI, we explored the relationship between changes in intracranial pressure and brain oxygenation (FIG. 5). From these studies it was found that a linear relationship exists between these two variables when ICP is between 10 and 25 mmHg. Above an ICP of 25 mm Hg, the brain is clearly maximally oxygen deprived. Current clinical practice does not suggest any intervention for brain swelling until ICP is between 20 and 25 mm Hg. However, our results indicate that, in order to ensure that adequate brain oxygenation occurs in all head injured patients, intervention to manage ICP should be initiated when ICP rises above 10 mm Hg. Once ICP starts to rise, any reduction of ICP with an NK1 antagonist will result in improved oxygenation of brain tissue, and hence have the potential to attenuate secondary brain injury.

The above experimental results support our hypothesis that substance P receptor antagonists are able to prevent and/or reverse the functional changes in the cerebral vasculature that lead to raised intracranial pressure. As such, administration of these agents enables the reduction of raised intracranial pressure. In turn this prevents the reduced blood and oxygenation supply to the brain, as well as mechanical damage to the brain tissue caused by herniation, which are central to poor patient prognosis. Our data supports the claim that substance P receptor antagonists represent an effective therapeutic intervention for reducing ICP, and thereby prevent or reduce the mortality and morbidity associated with this severe complication.

### Example 7

### Treatment with a NK1 Antagonist in a Rat Model of Traumatic Brain Injury

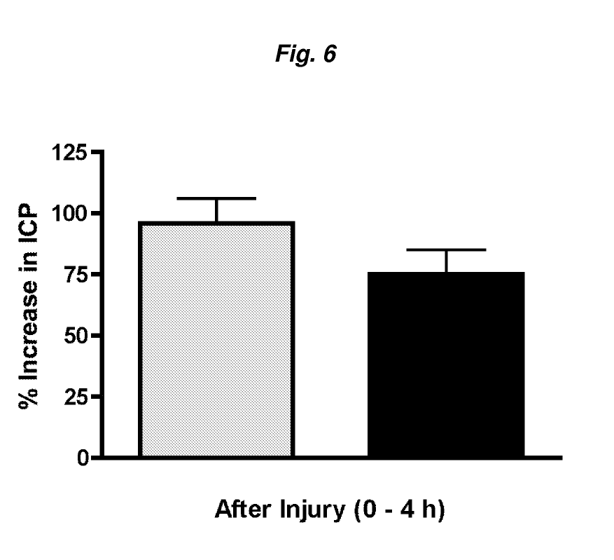

The effects of the NK1 antagonist on ICP after trauma were also examined in a second species, the rat. Male Sprague Dawley rats were injured using the widely accepted diffuse traumatic brain injury model, and then made hypoxic for a period of 15 minutes, before being ventilated on normal air. ICP was monitored using a Codman ICP pressure sensor for the ensuing 4 hours. After injury, ICP increased from a mean of 4.88±0.12 mm Hg to a mean of 9.23±0.28 mm Hg, an increase of 96%. Treatment with the NK1 antagonist, NAT, reduced ICP by a mean value 21% (FIG. 6). This decrease in ICP resulted in a significant improvement in functional outcome in all treated animals relative to vehicle treated controls.

Finally, it will be appreciated that various modifications and variations of the described methods and compositions of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are apparent to those skilled in the art are intended to be within the scope of the present invention.

1. A method of reducing intracranial pressure in a subject, the method including administering to the subject an effective amount of a substance P receptor antagonist.

2. The method according to claim 1, wherein the substance P receptor antagonist is a NK1 receptor antagonist, a NK2 receptor antagonist, or a NK3 receptor antagonist.

3.-5. (canceled)

6. The method according to claim 1, wherein the subject is susceptible to raised intracranial pressure.

7. The method according to claim 1, wherein the subject is suffering from raised intracranial pressure.

8. The method according to claims 6, wherein the raised intracranial pressure is a consequence of a stroke, acute brain injury, brain trauma, neurosurgical intervention, cranial haemorrhaging, cytotoxic and/or vasogenic oedema, peritumoural oedema, ischaemia, an infection of the central nervous system, neoplasia, liver failure, chronic obstructive lung disease, malaria, altitude sickness, and obstructive hydrocephalus.

9. The method according to claim 1, wherein the substance P receptor antagonist is administered to the subject at a dose of 0.25 mg/kg to 25 mg/kg.

10. (canceled)

11. A method of preventing and/or treating raised intracranial pressure in a subject, the method including administering to the subject an effective amount of a substance P receptor antagonist.

12. The method according to claim 11, wherein the substance P receptor antagonist is a NK1 receptor antagonist, a NK2 receptor antagonist, or a NK3 receptor antagonist.

13.-17. (canceled)

18. The method according to claim 11, wherein the raised intracranial pressure as a consequence of a stroke, acute brain injury, brain trauma, neurosurgical intervention, cranial haemorrhaging, cytotoxic and/or vasogenic oedema, peritumoural oedema, ischaemia, an infection of the central nervous system, neoplasia, liver failure, chronic obstructive lung disease, malaria, altitude sickness, and obstructive hydrocephalus.

19. The method according to claim 11, wherein the substance P receptor antagonist is administered to the subject at a dose of 0.25 mg/kg to 25 mg/kg.

20. (canceled)

21. The method according to claim 1, wherein blood flow to the brain of the subject is improved.

22.-28. (canceled)

29. The method according to claim 1, wherein oxygen delivery to the brain of the subject, and/or oxygenation of the brain of the subject is improved.

30.-36. (canceled)

37. The method according to claim 1, wherein risk of mortality or morbidity in the subject is reduced.

38.-44. (canceled)

45. The method according to claim 1, wherein risk of a neurological complication due to raised intracranial pressure in the subject is reduced.

46.-52. (canceled)

53. The method according to claim 1, wherein movement of water from the brain of the subject into the circulation is improved.

54.-62. (canceled)

63. The method according to claim 1, wherein the subject suffers from raised intracranial pressure, and wherein the prognosis or outcome of the subject is improved.

64.-71. (canceled)

72. The method according to claim 7, wherein the raised intracranial pressure is a consequence of a stroke, acute brain injury, brain trauma, neurosurgical intervention, cranial haemorrhaging, cytotoxic and/or vasogenic oedema, peritumoural oedema, ischaemia, an infection of the central nervous system, neoplasia, liver failure, chronic obstructive lung disease, malaria, altitude sickness, and obstructive hydrocephalus.
